(19) United States
(12) Patent Application Publication
(10) Pub. No.: US 2019/0142719 A9
(43) Pub. Date: May 16, 2019

(54) COSMETIC COMPOSITION COMPRISING A JASMONIC ACID COMPOUND

(71) Applicant: L'OREAL, Paris (FR)

(72) Inventors: Cecile Kermorvan, Creteil (FR); Magali Szestak, Boissise Le Bertrand (FR)

(21) Appl. No.: 15/367,847

(22) Filed: Dec. 2, 2016

Related U.S. Application Data
(63) Continuation of application No. 13/518,261, filed on Sep. 4, 2012, now abandoned.
(60) Provisional application No. 61/290,984, filed on Dec. 30, 2009.

(30) Foreign Application Priority Data
Dec. 22, 2009 (FR) 0959356

(51) Int. Cl. A61K 8/365; A61K 8/90; A61Q 19/00; A61Q 17/04; A61K 8/06

(52) U.S. Cl. CPC A61K 8/365; A61K 8/90; A61K 2800/48; A61Q 17/04; A61K 8/064; A61Q 19/00

(57) ABSTRACT

The invention relates to a cosmetic composition comprising, in a physiologically acceptable aqueous medium, a jasmonic acid compound and a non-ionic associative polyurethane/polyether polymer. Use for caring for and making up keratin materials.

### CROSS REFERENCE TO RELATED APPLICATIONS

This application is a Continuation of U.S. application Ser. No. 13/518,261 filed on Sep. 4, 2012, which is the National Phase filing under 35 U.S.C. § 371 of PCT/FR2010/052711 filed on Dec. 14, 2010; and this application claims priority to Application No. 0959356 filed in France on Dec. 22, 2009 and claims the benefit of U.S. Provisional Application No. 61/290,984 filed on Dec. 30, 2009 under 35 U.S.C. § 119. The entire contents of each application are hereby incorporated by reference.

The present invention relates to compositions, in particular cosmetic and/or dermatological compositions, comprising a jasmonic acid compound and a non-ionic associative polyurethane/polyether polymer, and also to the use of these compositions in a method for treating human keratin materials.

More particularly, the compositions of the invention are intended for caring for and/or making up keratin materials.

Within the meaning of the invention, the term “keratin materials” is intended to denote, for example, the skin, mucous membranes, the lips, the scalp, the eyelashes, the eyebrows and the hair.

Application EP-A-1 333 021 discloses jasmonic acid compounds, such as 3-hydroxy-2-pentylcyclopentaneacetic acid, for promoting desquamation of the skin and stimulating epidermal renewal, combating the signs of ageing of the skin, improving the radiance of the complexion and/or smoothing the skin of the face. In Application FR-A-62921255, these compounds are also described for their use as depigmenting agents.

Unfortunately, the introduction of the abovementioned jasmonic acid compounds into an aqueous cosmetic formulation can result in a not insignificant decrease in viscosity, thus inducing substantial fluidization of the composition.

A composition which is too fluid is difficult to apply to keratin materials. Such a composition runs on keratin materials, in particular on the skin, onto which it is applied. Its application to the keratin materials that it is desired to treat lacks precision and therefore makes it relatively unattractive to use.

In addition, the presence of a jasmonic acid compound is found to affect the thickening capacity of certain conventional gelling agents.

Alternative solutions consisting in compensating for this fall in viscosity by the addition of fatty alcohol and/or wax(es) do not prove to be satisfactory. The formulations thus obtained are generally too thick and whitening, are difficult to apply on and in particular to spread over keratin materials, and confer a feeling of heaviness, of difficulty in penetrating during application to the skin.

Thus, there exists a need to have available jasmonic acid compounds which can nevertheless have a significant thickening, if need be.

There also exists a need to have available cosmetic or dermatological compositions exhibiting a viscosity appropriate to ready application to keratin materials and in particular fluid compositions with a high content of particles which remain readily vaporizable.

The object of the present invention is, precisely, to satisfy these needs.

More specifically, the present invention relates to a composition comprising, in a physiologically acceptable medium comprising an aqueous medium, at least one jasmonic acid compound of formula (I) and at least one non-ionic associative polyurethane/polyether polymer.

The composition according to the invention is in particular a cosmetic and/or pharmaceutical composition, in particular a dermatological composition.

Surprisingly, the inventors have observed that the addition of a jasmonic acid compound to a composition comprising a non-ionic associative polyurethane/polyether polymer did not significantly affect the viscosity of the said composition and thus enabled it to be formulated in a form that is suited to its handling during its application. In particular, the viscosity of the composition does not fall.

What is more, this specific combination, jasmonic acid compound/non-ionic associative polyurethane/polyether polymer, also makes it possible to confer, on a composition of the invention, a pleasant appearance and, during the application thereof, comfortable feeling properties.

According to yet another of its subject-matters, the present invention relates to a non-therapeutic treatment method for caring for or making up keratin materials, comprising the application to the said keratin materials of a composition in accordance with the invention.

The compound derived from jasmonic acid is a compound chosen from those corresponding to the following formula (I):

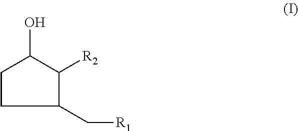

in which:

R₁ represents a COOR₃ radical, R₃ denoting a hydrogen atom or a C₁-C₄ alkyl radical, optionally substituted by one or more hydroxyl groups;

R₂ represents a saturated or unsaturated and linear hydrocarbon radical containing from 1 to 18 carbon atoms or a saturated or unsaturated and branched or cyclic hydrocarbon radical containing from 3 to 18 carbon atoms;

and also the optical isomers thereof, and corresponding salts.

Preferably, R₁ denotes a radical chosen from —COOH, —COOMe, —COO—CH₂—CH₃, —COO—CH₂—CH(OH)—CH₂OH, —COOCH₂—CH₂—CH₂OH or —COOCH₂—CH(OH)—CH₃. Preferentially, R₁ denotes a —COOH radical.

Preferentially, R₂ denotes a saturated or unsaturated and linear hydrocarbon radical preferably containing from 2 to 7 carbon atoms. In particular, R₂ may be a pentyl, pentenyl, hexyl or heptyl radical.

According to one embodiment, the compound of formula (I) is chosen from 3-hydroxy-2-[(2Z)-2-pentenyl]cyclopentaneacetic acid or 3-hydroxy-2-pentylcyclopentaneacetic acid. Preferably, compound (I) is 3-hydroxy-2-pentylcyclopentaneacetic acid; this compound may especially be in the form of the sodium salt.

The salts of the compounds that may be used according to the invention are chosen in particular from salts of alkali metals, for example sodium or potassium; salts of alkaline-earth metals, for example calcium, magnesium or strontium; metal salts, for example zinc, aluminium, manganese or copper; salts of ammonium of formula NH₄⁺; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts. Salts chosen from sodium, potassium, magnesium, strontium, copper, manganese or zinc salts are preferably used. The sodium salt is preferentially used.

The compound of formula (I) defined previously may be present in the composition according to the invention in a content ranging from 0.01% to 10% by weight and preferably from 0.1% to 5% by weight, relative to the total weight of the composition.

The composition according to the invention comprises an associative polyurethane/polyether polymer.

Within the meaning of the present invention, the term “associative polymers” is understood to mean hydrophilic polymers that are capable, in an aqueous medium, of reversibly combining with one another or with other molecules. Their chemical structure more particularly comprises at least one hydrophilic region and at least one hydrophobic region.

The term “hydrophobic group” is understood to mean a radical or polymer comprising a saturated or unsaturated and linear or branched hydrocarbon chain. When the hydrophobic group denotes a hydrocarbon radical, it comprises at least 10 carbon atoms, preferably from 10 to 30 carbon atoms, in particular from 12 to 30 carbon atoms and more preferably from 18 to 30 carbon atoms. Preferentially, the hydrocarbon group is derived from a monofunctional compound.

By way of example, the hydrophobic group may be derived from a fatty alcohol, such as stearyl alcohol, dodecyl alcohol or decyl alcohol, or else from a polyalkylenated fatty alcohol, such as Steareth-100. It may also denote a hydrocarbon polymer, for instance polybutadiene.

The non-ionic polyurethane/polyethers according to the invention generally comprise, in their chain, both hydrophilic blocks, usually of polyoxyethylene nature, and hydrophobic blocks that may be aliphatic sequences alone and/or cycloaliphatic and/or aromatic sequences.

Preferably, these polyurethane/polyethers comprise at least two lipophilic hydrocarbon chains containing from 6 to 30 carbon atoms, separated by a hydrophilic block, the hydrocarbon chains possibly being pendent chains or chains at the end of the hydrophilic block. In particular, it is possible for one or more pendent chains to be provided. In addition, the polymer may comprise a hydrocarbon chain at one end or at both ends of a hydrophilic block.

The polyurethane/polyethers may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The non-ionic polyurethane/polyethers comprising a fatty chain may be triblock copolymers, the hydrophilic block of which is a polyoxyethylene chain comprising from 50 to 1000 oxyethylene groups.

The non-ionic polyurethane/polyethers comprise a urethane linkage between the hydrophilic blocks, whence arises the name.

By extension, also included among the non-ionic polyurethane/polyethers comprising a hydrophobic chain are those in which the hydrophilic blocks are linked to the hydrophobic blocks via other chemical bonds.

As examples of non-ionic polyurethane/polyethers comprising a hydrophobic chain that may be used in the invention, it is also possible to use Rheolate 205® containing a urea functional group, sold by the company Rheox, or Rheolate® 208, 204 or 212, and also Acrysol RM 184®.

Mention may also be made of the product Elfacos T210® containing a C₁₂-C₁₄ alkyl chain, and the product Elfacos T212® containing a C₁₈ alkyl chain, from Akzo.

The product DW 1206B® from Rohm & Haas containing a C₂₀ alkyl chain and a urethane linkage, sold at a solids content of 20% in water, may also be used.

It is also possible to use solutions or dispersions of these polymers, in particular in water or in aqueous/alcoholic medium. Examples of such polymers that may be mentioned are Rheolate® 255, Rheolate® 278 and Rheolate® 244 sold by the company Rheox. The products DW 1206F and DW 1206J, sold by the company Rohm & Haas, may also be used.

The polyurethane/polyethers that may be used according to the invention may also be chosen from those described in the paper by G. Fonnum, J. Bakke and Fk. Hansen, Colloid Polym. Sci., 271, 380-389 (1993).

According to a specific form of the invention, use will be made of a polyurethane/polyether that may be obtained by polycondensation of at least three compounds comprising (i) at least one polyethylene glycol comprising from 150 to 180 mol of ethylene oxide, (ii) a polyoxyethylenated stearyl alcohol comprising 100 mol of ethylene oxide, and (iii) a diisocyanate.

Such polyurethane/polyethers are sold especially by the company Elementis under the name Rheolate FX 1100®, which is a polycondensate of polyethylene glycol containing 136 mol of ethylene oxide, of stearyl alcohol polyoxyethylenated with 100 mol of ethylene oxide and of hexamethylene diisocyanate (HDI) with a weight-average molecular weight of 30 000 (INCI name: PEG-136/Steareth-100/HDI Copolymer).

According to another specific form of the invention, use will be made of a polyurethane/polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane/polyethers are sold in particular by the company Rohm & Haas under the names Aculyn 46® and Aculyn 44®.

Aculyn 46® having the INCI name: PEG-150/Stearyl Alcohol/SMDI Copolymer, is a polycondensate of polyethylene glycol comprising 150 or 180 mol of ethylene oxide, of stearyl alcohol and of methylenebis(4-cyclohexyl isocyanate) (SMDI) at 15% by weight in a matrix of maltodextrin (4%) and water (81%) (INCI name: PEG-150/Stearyl Alcohol/SMDI Copolymer).

Aculyn 44® (PEG-150/Decyl Alcohol/SMDI Copolymer) is a polycondensate of polyethylene glycol comprising 150 or 180 mol of ethylene oxide, of decyl alcohol and of methylenebis(4-cyclohexyl isocyanate) (SMDI) at 35% by weight in a mixture of propylene glycol (39%) and water (26%) (INCI name: PEG-150/Decyl Alcohol/SMDI Copolymer).

The non-ionic associative polyurethane/polyether polymer described above may be present in the composition according to the invention in a content ranging from 0.05% to 1% by weight, preferably ranging from 0.1% to 0.6% by weight and preferentially ranging from 0.1% to 0.5% by weight, relative to the total weight of the composition.

Advantageously, the jasmonic acid compound of formula (I) and the non-ionic associative polyurethane/polyether polymer which are described above can be present in the composition according to the invention in a compound (I)/non-ionic associative polyurethane/polyether polymer ratio by weight ranging from 30 to 60 and preferably ranging from 40 to 50.

Protocol for Measuring the Viscosity:

The viscosity of a composition of the invention may be measured via any method known to those skilled in the art, and especially according to the following conventional method. Thus, the measurement can be carried out at 25° C. using a Contraves TV or Rheomat 180 equipped with a spindle rotating at 200 rpm. Those skilled in the art may select the spindle for measuring the viscosity from the spindles M1 or M2 on the basis of their general knowledge, so as to be able to perform the measurement.

The addition of a jasmonic acid compound as defined above to an aqueous solution comprising the terpolymer according to the invention is reflected by a variation in viscosity of less than 20%, measured with respect to the viscosity of a solution comprising only the polymer, in particular of less than or equal to 15% and in particular of less than or equal to 10%.

The composition according to the invention comprises a physiologically acceptable aqueous medium.

The term “physiologically acceptable medium” is intended to denote a medium that is compatible with human keratin materials and/or fibres, for instance, in a non-limiting manner, the skin, the mucous membranes, the nails, the scalp and/or the hair.

This physiologically acceptable aqueous medium comprises an aqueous phase, optionally as a mixture, or not, with one or more organic solvents, such as a C₁-C₈ alcohol, in particular ethanol, isopropanol, tert-butanol, n-butanol, polyols, such as glycerol, propylene glycol or butylene glycol, and polyol ethers.

A composition according to the invention may also comprise a fatty phase, which may comprise oils, gums and waxes normally used in the field of application under consideration.

Thus, according to one embodiment, a composition according to the invention may also comprise at least one fatty phase chosen from a fatty phase which is solid at ambient temperature (20-25° C.) and atmospheric pressure and/or a fatty phase which is liquid at ambient temperature (20-25° C.) and atmospheric pressure.

A liquid fatty phase suitable for implementing the invention may comprise a volatile oil, a non-volatile oil, and a mixture thereof. A volatile or non-volatile oil may be a hydrocarbon oil, in particular of animal or vegetable origin, a synthetic oil, a silicone oil, a fluoro oil or a mixture thereof.

A solid fatty phase suitable for implementing the invention may be, for example, chosen from pasty fatty substances, gums, and mixtures thereof.

As oils or waxes that may be used in the invention, mention may be made of mineral oils (liquid petroleum jelly), vegetable oils (liquid fraction of shea butter, sunflower oil), animal oils (perhydrosqualene), synthetic oils (Purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils.

When a composition is an emulsion, the proportion of the fatty phase may range from 5% to 80% by weight and preferably from 5% to 50% by weight relative to the total weight of the composition. The oils, waxes, emulsifiers and co-emulsifiers used in the composition in emulsion form are chosen from those conventionally used in the cosmetics field.

An emulsifier and a co-emulsifier may be present in a composition of the invention in a proportion ranging from 0.3% to 30% by weight and in particular from 0.5% to 20% by weight relative to the total weight of the composition.

An emulsion according to the invention may also contain lipid vesicles.

When a composition according to the invention is an oily solution or gel, the fatty phase may represent more than 90% of the total weight of the composition.

A composition according to the invention may also contain adjuvants that are common in the field under consideration, such as surfactants, emulsifiers, hydrophilic or lipophilic gelling agents, hydrophilic or lipophilic additives, preservatives, antioxidants, solvents, fragrances, fillers, UV-A and/or UV-B screening agents (organic or inorganic, soluble or insoluble), pigments, fibres, chelating agents, odour absorbers, colourants and other cosmetic or pharmaceutical active agents.

The amounts of these various adjuvants are those conventionally used in the cosmetics field, and may range, for example, from 0.01% to 30% of the total weight of the composition. In general, the amounts are adjusted as a function of the formulation prepared. Depending on their nature, these adjuvants may be introduced into the fatty phase, into the aqueous phase and/or into the lipid spherules.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, mention may be made of modified clays such as bentones, metal salts of fatty acids, for instance aluminium stearates, and hydrophobic silica.

A composition of the invention can be provided in all the formulation forms which can be envisaged.

In particular, a composition according to the invention may have the form of an aqueous or aqueous/alcoholic solution; a dispersion; a water-in-oil, oil-in-water or multiple emulsion; a suspension; microcapsules or microparticles; vesicular dispersions of ionic and/or non-ionic type; or an aerosol composition also comprising a pressurized propellant. Preferentially, the composition according to the invention may be an oil-in-water emulsion.

A composition according to the invention can be provided in the form of a hair-care composition, in particular a shampoo, a hair-setting lotion, a treating lotion, a styling cream or gel, a dyeing composition, in particular an oxidation dyeing composition, hair-restructuring lotions, a perming composition (in particular a composition for the first step of a perming), a lotion or a gel for combating hair loss, or an antiparasitic shampoo.

It can also be provided in the form of a cleansing, protecting, treating or caring composition for the face, for the hands, for the feet, for the major anatomical folds or for the body (for example, day cream, night cream, make-up-removing cream, anti-sun composition, protective or care body milk, after-sun milk, lotion, gel or foam for caring for the skin, such as cleansing lotions, or artificial tanning compositions); a composition for making up the body or face, such as a foundation; a bath composition; a deodorizing composition comprising, for example, a bactericidal agent; an aftershave composition; a depilatory composition; a composition for countering insect stings or bites; a pain-relieving composition; or a dermatological or pharmaceutical composition for treating certain skin diseases, such as eczema, rosacea, psoriasis, lichen or severe pruritus.

When a composition according to the invention is intended for a use of peeling type, it can also be provided in all the formulation forms mentioned above, provided that it is easily removed by rinsing, in particular in the form of an aqueous gel or an aqueous or aqueous/alcoholic solution.

A composition according to the invention can be applied by any means which makes possible uniform distribution and in particular using a cotton wool swab, a rod, a brush, a gauze, a spatula or a pad, or else by spraying, and can be removed by rinsing with water or using a mild detergent.

A composition according to the invention can be provided in a fluid form of vaporizable or non-vaporizable liquid type, in the form of a paste, of a direct or inverse emulsion, or of an impregnated support or gel.

In particular, a composition according to the invention can be provided in a solid form, in particular a compact, pulverulent or cast solid form, or in a stick form.

A composition according to the invention may also be in the form of a care product, an anti-sun or after-sun product, a daily photoprotective care product, a body product, a foundation to be applied to the face or the neck, a concealer product, a complexion corrector, a tinted cream or a make-up base for making up the face, or a body make-up composition.

A composition according to the invention may be used for the purposes of improving the general condition of the epidermis, in particular of the skin, and especially for maintaining or restoring its physiological functions and/or its aesthetic appearance.

Thus, a composition according to the invention can advantageously be employed in order to combat ageing of the epidermis, to maintain and/or stimulate the moisturizing and/or to combat the drying out of the skin, to improve the tonicity of the skin, to maintain or restore the suppleness and elasticity of the skin, to improve the mineralization of the epidermis, to improve the vitality of the epidermis, to facilitate intercellular exchanges, and to combat chapping and the cracked appearance of the skin.

A composition according to the invention may be intended for a cosmetic and/or dermatological application.

Other characteristics and advantages of the invention will emerge more clearly from the examples that follow, which are given as non-limiting illustrations. In the text hereinbelow or hereinabove, the proportions are given as weight percentages, unless otherwise indicated.

### COMPARATIVE EXAMPLES 1 TO 4

An aqueous gel was produced with three associative thickening polymers (two non-ionic associative polyurethane/polyether polymers according to the invention and one associative polymer outside the invention) and, for each of the polymers, the gel was produced with or in the absence of sodium salt of 3-hydroxy-2-pentylcyclopentaneacetic acid.

The viscosity of the aqueous gels obtained was then measured after 24 hours of storage at ambient temperature (viscosity measured at 25° C. using a Contraves TV with an M3 spindle after 10 minutes of rotation at 200 revolutions/minute).

| | | Example 1B |
|---|---|---|
| | Example 1A | (invention) |
| Sodium salt of 3-hydroxy-2- | 0 | 3.3%, i.e. |
| pentylcyclopentaneacetic acid at | | 1% AM |
| 30% in a water/dipropylene glycol | | |
| (70/30) mixture | | |
| PEG-136/Steareth-100/HDI | 3% | 3% |
| Copolymer (Rheolate FX 1100 | | |
| from Elementis) | | |
| Water | q.s. for | q.s. for |
| | 100% | 100% |
| Viscosity (Pa · s) | 1.11 | 1.57 |
| | | Example 2B |
| | Example 2A | (invention) |
| Sodium salt of 3-hydroxy-2- | 0 | 2.98%, i.e. |
| pentylcyclopentaneacetic acid at | | 1% AM |
| 30% in a water/dipropylene glycol | | |
| (70/30) mixture | | |
| PEG-150/Decyl Alcohol/SMDI | 5% | 5% |
| Copolymer (Aculyn 44 from Rohm | | |
| & Haas) | | |
| Water | q.s. for | q.s. for |
| | 100% | 100% |
| Viscosity (Pa · s) | 0.2 | 0.35 |

| | | Example 3B |
|---|---|---|
| | | (outside the |
| | Example 3A | invention) |
| Sodium salt of 3-hydroxy-2- | 0 | 2.98%, i.e. |
| pentylcyclopentaneacetic acid at | | 1% AM |
| 30% in a water/dipropylene glycol | | |
| (70/30) mixture | | |
| Ammonium | 2% | 2% |
| Acryloyldimethyltaurate/Steareth-8 | | |
| Methacrylate Copolymer (Aristoflex | | |
| SNC from Clariant) | | |
| Water | q.s. for | q.s. for |
| | 100% | 100% |
| Viscosity (Pa · s) | 7.1 | 5.2 |
| | | Example 4B |
| | | (outside the |
| | Example 4A | invention) |
| Sodium salt of 3-hydroxy-2- | 0 | 2.98%, i.e. |
| pentylcyclopentaneacetic acid at | | 1% AM |
| 30% in a water/dipropylene glycol | | |
| (70/30) mixture | | |
| Acrylamide/sodium acrylamido-2- | 3% | 3% |
| methylpropanesulphonate | | |
| copolymer as an inverse emulsion | | |
| at 40% in isoparaffin/water (Sepigel | | |
| 305 from Seppic) | | |
| Water | q.s. for | q.s. for |
| | 100% | 100% |
| Viscosity (Pa · s) | 2.93 | 1.45 |

These tests show that only the gels comprising the non-ionic associative polyurethane/polyethers Rheolate FX1100 and Aculyn 44 in the presence of the sodium salt of 3-hydroxy-2-pentylcyclopentaneacetic acid do not exhibit a loss in viscosity, while the gel comprising Aristoflex SNC in the presence of the same active agent exhibits a fall in viscosity of approximately 26% and the gel comprising Sepigel 305 exhibits a fall in viscosity of approximately 50%.

Thus, the presence of non-ionic associative polyurethane/polyether gum makes it possible to maintain the viscosity of the aqueous gel in the presence of the sodium salt of 3-hydroxy-2-pentylcyclopentaneacetic acid.

### EXAMPLE 5

A cream for the care of the face having the following composition was prepared:

| | % |
|---|---|
| Compounds | weight |
| PEG-136/Steareth-100/HDI Copolymer (Rheolate FX 1100 | 0.7 |
| from Elementis) | |
| Sodium salt of 3-hydroxy-2-pentylcyclopentaneacetic | 3% AM |
| acid at 30% in a water/dipropylene glycol (70/30) mixture | |
| Water | q.s. for |
| | 100 |
| Methyl glucose sesquistearate (Glucamate SS from Noveon) | 2 |
| Oxyethylenated methyl glucose sesquistearate | 2 |
| (Glucamate SSE 20 from Noveon) | |
| Cyclohexasiloxane | 10 |
| Apricot kernel oil | 6 |
| Shorea butter | 2 |
| Preservative | 0.3 |

The composition obtained has the appearance of a thick cream which melts during its application to the skin.

The composition applied to the face makes it possible to revive the radiance of the complexion.

### EXAMPLE 6

A vaporizable anti-sun fluid is prepared:

| COMPOUNDS | Ex. A | Ex. B |
|---|---|---|
| Sequestering agent | q.s. | q.s. |
| Triethanolamine | q.s. | q.s. |
| Butyl Methoxydibenzoylmethane (4-tert-butyl- | 1 | 3 |
| 4′-methoxydibenzoylmethane) (Parsol 1789 | | |
| from DSM Nutritional Products) | | |
| 2-Ethylhexyl salicylate (Neoheliopan OS from | 0 | 5 |
| Symrise) | | |
| Ethylhexyl Triazone (Uvinul T 150 from BASF) | 0.5 | 0.5 |
| Terephthalylidene Dicamphor Sulfonic Acid (Mexoryl | 1.5 | 1.5 |
| SX from Chimex) | | |
| Octocrylene (Uvinul N 539 from BASF) | 10 | 10 |
| Titanium dioxide (MT100 AQ from Tayca) | 0 | 3.5 |
| Diglycol/Cyclohexanedimethanol/Isophthalates/ | 2 | 2 |
| Sulfoisophthalates Copolymer (Eastman AQ 38S | | |
| polymer from Eastman Chemical) | | |
| PEG-150/Decyl Alcohol/SMDI Copolymer | 1 | 1.3 |
| (Aculyn 44 from Rohm & Haas) | | |
| Cyclohexasiloxane | 5 | 5 |
| Water | q.s. for | q.s. for |
| | 100% | 100% |
| C12-C15 Alkyl benzoate (Finsolv TN from Finetex) | 6 | 1 |
| Glycerin | 3 | 3 |
| Sodium salt of 3-hydroxy-2-pentylcyclopentaneacetic | 1% AM | 1% AM |
| acid at 30% in a water/dipropylene glycol (70/30) | | |
| mixture | | |
| Preservative | q.s. | q.s. |

The products obtained are stable and sprayable and confer anti-sun protection on the skin, in addition to the biological activity contributed by the active agent.

1. A composition comprising, in a physiologically acceptable aqueous medium, a compound of following formula (I): 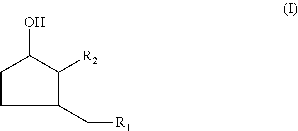
in which:
R₁ represents a COOR₃ radical, R₃ denoting a hydrogen atom or a C₁-C₄ alkyl radical, optionally substituted by one or more hydroxyl groups;
R₂ represents a saturated or unsaturated and linear hydrocarbon radical containing from 1 to 18 carbon atoms or a saturated or unsaturated and branched or cyclic hydrocarbon radical containing from 3 to 18 carbon atoms;
and the optical isomers thereof, and the corresponding salts;
and a non-ionic associative polyurethane/polyether polymer.

2. The composition according to claim 1, wherein R₁ denotes a radical selected from the group consisting of —COOH, —COOMe, —COO—CH₂—CH₃,
—COO—CH₂—CH(OH)—CH₂OH, —COOCH₂—CH₂—CH₂OH and —COOCH₂—CH(OH)—CH₃, —COO—CH₂—CH₃;
R₂ denotes a saturated or unsaturated and linear hydrocarbon radical containing from 2 to 6 carbon atoms.

3. The composition according to claim 1, wherein compound (I) is 3-hydroxy-2-pentylcyclopentaneacetic acid.

4. The composition according to claim 1, wherein the compound of formula (I) is present in a content ranging from 0.01% to 10% by weight, relative to the total weight of the composition.

5. The composition according to claim 1, wherein the non-ionic associative polyurethane/polyether comprises at least two lipophilic hydrocarbon chains containing from 6 to 30 carbon atoms, separated by a hydrophilic block, the hydrocarbon chains possibly being pendent chains or chains at the end of the hydrophilic block.

6. The composition according to claim 1, wherein the non-ionic associative polyurethane/polyether is in triblock form.

7. The composition according to claim 1, wherein the non-ionic associative polyurethane/polyether is in triblock form, the hydrophilic block of which is a polyoxyethylene chain comprising from 50 to 1000 oxyethylene groups.

8. The composition according to claim 1, wherein the non-ionic associative polyurethane/polyether is obtained by polycondensation of at least three compounds comprising (i) at least one polyethylene glycol comprising from 150 to 180 mol of ethylene oxide, (ii) a polyoxyethylenated stearyl alcohol comprising 100 mol of ethylene oxide, and (iii) a diisocyanate.

9. The composition according to claim 1, wherein the non-ionic associative polyurethane/polyether is a PEG-136/Steareth-100/HDI copolymer.

10. The composition according to claim 1, wherein the non-ionic associative polyurethane/polyether is obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

11. The composition according to claim 1, wherein the non-ionic associative polyurethane/polyether is a PEG-150/Stearyl Alcohol/SMDI copolymer or a PEG-150/Decyl Alcohol/SMDI copolymer.

12. The composition according to claim 1, wherein the non-ionic associative polyurethane/polyether polymer is present in a content ranging from 0.05% to 1% by weight, relative to the total weight of the composition.

13. The composition according to claim 1, wherein it is in the form of a water-in-oil emulsion.

14. A method for the non-therapeutic treatment of keratin materials, comprising the application to the said keratin materials of a cosmetic composition as defined according to claim 1.

15. The composition according to claim 2, wherein compound (I) is 3-hydroxy-2-pentylcyclopentaneacetic acid.

16. The composition according to claim 1, wherein compound of formula (I) is present in a content ranging from 0.1% to 5% by weight, relative to the total weight of the composition.

17. The composition according to claim 1, wherein the non-ionic associative polyurethane/polyether polymer is present in a content ranging from 0.1% to 0.6% by weight relative to the total weight of the composition.

18. The composition according to claim 1, wherein the non-ionic associative polyurethane/polyether polymer is present in a content ranging from 0.1% to 0.5% by weight relative to the total weight of the composition.

19. The composition according to claim 2, wherein the compound of formula (I) is present in a content ranging from 0.01% to 10% by weight relative to the total weight of the composition.

20. The composition according to claim 3, wherein the compound of formula (I) is present in a content ranging from 0.01% to 10% by weight relative to the total weight of the composition.
